(19) United States
(12) Patent Application Publication
(10) Pub. No.: US 2010/0168031 A1
(43) Pub. Date: Jul. 1, 2010

(54) Lat peptides and their use in assays for identifying immunosuppressants

(76) Inventor: Francois Cardinaux, Seewan (CH)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 10/344,201

(22) PCT Filed: Aug. 7, 2001
(86) PCT No.: PCT/EP01/09134

§ 371 Date: Aug. 11, 2003

(30) Foreign Application Priority Data
Aug. 9, 2000 (GB) 0019632.9

(51) Int. Cl. A61K 38/10; C07K 7/08; C07K 7/06; G01N 33/573; A61K 38/08

(52) U.S. Cl. 514/14; 530/326; 530/328; 435/7.4; 514/16

(57) ABSTRACT

The invention relates to novel peptides and their use in assays for identifying novel immunosuppressants. More particularly the present invention provides methods and compositions for identifying compounds that will modulate the interaction of protein tyrosine kinase substrates with their intracellular ligands, as well as between their intracellular ligands and other members of the signaling pathway.

The present invention relates to novel peptides and their use in assays for identifying novel immunosuppressants. In particular, the present invention contemplates screening compounds and identifying compounds that modulate the interaction of the Syk family protein tyrosine kinases Syk and ZAP-70, and their substrates.

T cells play a key role in transplant rejection, autoimmune diseases and the initiation of inflammatory responses and are thus a primary target for pharmaceutical intervention in these indications. The activation of T cells is a complex process which results in cell growth and differentiation. The engagement of the T cell receptor on mature peripheral T cells initiates multiple intracellular signals that lead to cellular proliferation and the acquisition of complex functions. The biochemical mechanisms that couple receptor binding to these intracellular events have been intensively investigated (Van Leeuwen, J. E. M. and Samelson, L. E., Curr. Opin. Immunol. 11, 242-248, 1999) The Syk family of protein tyrosine kinases, comprising Syk and ZAP-70, play an essential role in the initiation and the amplification of receptor signal transduction (Chu, D. H. et al., Immunol. Rev. 165, 167-180, 1998). ZAP-70 is expressed solely in T cells and NK cells (natural killer cells), where it phosphorylates its specific substrate LAT (linker for activation of T cells), which then recruits a number of downstream effector molecules. Syk is found in B cells, mast cells, neutrophils, macrophages and platelets and is involved in B cell receptor and Fc receptor signal transduction.

In one embodiment, the present invention contemplates identifying compounds, which modulate the interaction of LAT and activated, phosphorylated ZAP-70 and/or Syk kinases, and which therefore act as immunosuppressants.

In preferred embodiments, disclosed LATs are useful in drug screening assays designed to identify drugs that interfere with the specific binding of activated ZAP-70 and/or Syk kinases with their substrates and thereby block further signal transduction.

In other embodiments, the invention provides LAT polypeptides having ZAP-70 and/or Syk kinase-specific binding affinity. The invention also provides nucleic acids encoding the disclosed LATs as part of expression vectors for introducing into cells.

The present invention provides in a first aspect a compound of formula I

R¹-Tyr-R² (I)

wherein

R¹ is HOOC(CH₂)₂CO— or R³-Asp- wherein R³ is hydrogen, an amino acyl residue or an oligopeptidyl residue; and

R² is an amino acid, preferentially a naturally occurring amino acid or an oligopeptide, preferentially an oligopeptide made of natural amino acids;

with the proviso that compounds represented by SEQ ID NO:1, SEQ ID NO:2, SEQ ID NO:3, and SEQ ID NO:4 are excluded.

The compounds of formula I may, in addition to the succinoyl-Tyr- or -Asp-Tyr motif, comprise any amino acid, preferentially a naturally occurring amino acid. The oligopeptidyl residue or oligopeptide may preferably be from 2 to 7 amino acid residues long. A more preferred number of amino acids may be from 5 to 7 amino acid residues long.

The compounds of formula I may be free or bound to either a solid support, e.g. polystyrene (e.g. poly-lysine coated polystyrene microtiter plates), polyamide (e.g. polyacrylamide pins or beads or cellulose, e.g. paper); a labeling group, e.g. biotin, in particular L(₊)-biotin (also others e.g. oligo-lysine or arginine (Lys and/or Arg)ₙ, n=2 to 6), a chromogenic or a fluorescent group (e.g. 2,4-dinitrophenyl); or a macromolecular carrier, e.g. another peptide, e.g. a membrane permeable peptide like penetratin or lipopeptide or a protein (e.g. bovine serum albumine). Attachment may be via any one of the side chains of the amino acids contained in the compound of formula I except of the tyrosine in the succinoyl-Tyr- or of the aspartic and tyrosine in the -Asp-Tyr- motif, or via the terminal amino or carboxy group, either directly or via a linker, e.g. aminohexanoyl or succinyl. The amino groups of compounds of formula I may be free or protected by any of the protecting groups known to a person skilled in the art of peptide chemistry, e.g. by an acyl, urethane or urea group, more preferably acetyl, benzyloxycarbonyl, tert.butyloxycarbonyl or 9-fluorenylmethoxycarbonyl (Fmoc). The terminal carboxy group may be replaced by a functional derivative, e.g. an ester, e.g. alkyl or aralkyl, e.g. methyl or benzyl ester, an unsubstituted amide or a mono- or di-substituted amide, e.g. N-methyl, N,N-diethyl or N-phenyl, N-ethyl amide.

Examples for compounds of formula I include compounds of formula IIa and IIb

R⁴—Y¹—(X⁶)ₐ(X⁵)_{b}(X⁴)_{c}(X³)_{d}(X²)ₑAspTyrX^{1′}(X^{2′})ᵣ(X^{3′})_{q}(X^{4′})ₚ(X^{5′})ₒ(X^{6′})ₙ—NHCH(R⁶)C(O)—R⁵ (IIa)

HOOC(CH₂)₂CO-Tyr-X^{1′}—(X^{2′})ᵣ—(X^{3′})_{q}—(X^{4′})ₚ—(X^{5′})ₒ(X^{6′})ₙ—NHCH(R⁶)C(O)—R⁵ (IIb)

wherein

each of X⁶, X⁵, X4, X³, X², X^{1′}, X^{2′}, X^{3′}, X^{4′}, X^{5′} and X^{6′} is an amino acid, preferentially a naturally occurring amino acid;

Y¹ is a linking group or single bond;
R⁴ is hydrogen, a labeling group, C₁-C₈alkoxycarbonyl, R^{a}—C(O)—, R^{a}—OC(O)— or R^{a}—NHC(O)— wherein R^{a} is C₁-C₈alkyl, carboxy-C₁-C₈alkyl, aryl or aralkyl wherein the aryl group is unsubstituted or substituted by halogen, C₁-C₄alkyl or C₁-C₈alkoxy;
R⁵ is NR⁷R⁸ or OR⁹ wherein R⁷ and R⁸ independently are hydrogen, C₁-C₈alkyl, or R⁷ and R⁸ together with the nitrogen atom to which they are attached form a heterocyclic residue wherein the heterocyclic residue is a five or six ring and preferentially in which a further residue in the ring may be N, O or S, and R⁹ is hydrogen or C₁-C₈alkyl;

R⁶ is an amino acid side chain; either

(a) a, b, c, d and e are 1; or

(b) a is 0 and b, c, d and e are 1; or

(c) a and b are 0 and c, d and e are 1; or

(d) a, b and c are 0 and d and e are 1; or

(e) a, b, c and d are 0 and e is 1; or

(f) a, b, c, d and e are 0; and either

(a′) n, o, p, q and r are 1; or

(b′) n is 0 and o, p, q and r are 1; or

(c′) n and o are 0 and p, q and r are 1; or

(d′) n, o and p are 0 and q and r are 1; or

(e′) n, o, p and q are 0 and r is 1; or

(f′) n, o, p, q and r are 0.

Aryl alkyl groups or moiety may be linear or branched.

In formula IIa the following significances are preferred independently, collectively or in any combination or sub-combination:

R⁴ is hydrogen, acetyl, succinoyl or L(+)-biotinyl;

Y¹ is aminohexanoyl, succinyl or a direct bond;

X⁶ is Glu, Ser or Gly, preferably Glu;

X⁵ is Glu, Ala or Phe, preferably Glu;

X⁴ is Asp, Glu, Gln, Ser, Gly or Leu, preferably Asp, Ser or Gly;

X³ is Glu, Gln, Asp, Ile, Ala or Asp, preferably Glu, Ile or Ala;

X² is Pro, Asp or Gly, preferably Pro or Asp;

X¹ is Glu, His, Val or Met, preferably Glu;

X^{2′} is Asn, Asp, Ser, Glu, Trp, Tyr or Phe, preferably Asn or Phe;

X^{3′} is Pro, Val, Leu, Met or Nle, preferably Pro, Val or Leu;

X^{4′} is Met, Gln, Ser, Pro, Asn, Gly or Nle, preferably Gly, Pro or Gln;

X^{5′} is Gln, Glu, Asp or Val, preferably Glu;

X^{6′} is Glu, Gln, Leu or Asp, preferably Leu;

R⁵ is —OH, —OCH₃, —OCH₂CH₃, —NH₂, NHCH₃, N(CH₃)₂, NHCH₂CH₃;

R⁶ is (CH₃)₂—CH—CH₂—, H₂N—CO—(CH₂)₂—, H₂N—CO—CH₂—, HOOC—CO—(CH₂)₂—, preferably H₂N—CO—CH₂—; and

a, b, c, d, e, n, o, p, q and r are as defined above.

In formula IIb the following significances are preferred independently, collectively or in any combination or sub-combination:

X^{1′} is Glu, His, Val or Met, preferably Glu;

X^{2′} is Asn, Asp, Ser, Glu, Trp, Tyr or Phe, preferably Asn or Phe;

X^{3′} is Pro, Val, Leu, Met or Nle, preferably Pro, Val or Leu;

X^{4′} is Met, Gln, Ser, Pro, Asn, Gly or Nle, preferably Gly, Pro or Gln;

X^{5′} is Gln, Glu, Asp or Val, preferably Glu;

X^{6′} is Glu, Gln, Leu or Asp, preferably Leu;

R⁵ is —OH, —OCH₃, —OCH₂CH₃, —NH₂, NHCH₃, N(CH₃)₂, NHCH₂CH₃;

R⁶ is the side chain of Leu, Gln, Asn or Glu, preferably Asn; and

n, o, p, q and r are as defined above.

Preferably R⁵ is —OH when n, o, p, q and r are 1, and NH₂ or NHCH₂CH₃ when n, n and o, n, o and p, n, o, p and q, or n, o, p, q and r are 0.

Preferred peptide sequences are SEQ ID NO:5, SEQ ID NO:6, SEQ ID NO:7, SEQ ID NO:8, SEQ ID NO:9, SEQ ID NO:10, and SEQ ID NO:24

wherein the N-terminal Xaa represents R⁴—Y¹— and the C-terminal Xaa represents —NHCH(R⁶)C(O)—R⁵, wherein R⁴, and R⁵ are as defined above, and R⁶ is the Asn side chain in the case of SEQ ID NO:5 and SEQ ID NO:8, the glycine side chain in the case of SEQ ID NO:6 and SEQ ID NO:7, and the leucine side chain in the case of SEQ ID NO:9, SEQ ID NO:10 and SEQ ID NO:24.

A more preferred peptide sequence is SEQ ID NO:5.

Preferred compounds of formula IIa and IIb are those wherein R⁵ is OH or NH₂, in particular OH, or Y¹ is a direct bond and R⁴ is —C(O)CH₃ or Y¹ is aminohexanoyl and R⁴ is biotinyl.

Most preferred compounds of formula I are those represented by SEQ ID NO:21, wherein Xaa is H₃CC(O)— and SEQ ID NO:12, wherein Xaa is L(+)-biotinyl-aminohexanoyl.

The compounds of formula I may be prepared in analogy with known methods, e.g. as known in the art of peptide synthesis or as described in the following examples. Where desired protecting groups known to the skilled artisan may temporarily be attached to one or more of the functional groups present. Protecting groups also include a polymer resin having suitable functional groups. Following a typical protocol, compounds of the invention may for example be synthesized in a stepwise manner on a resin support, e.g. a polystyrene based resin support. The α-amino group may, e.g., be protected by Fmoc and the side-chain functional groups may, e.g., be protected by tert.-butyl or triphenylmethane (Trt). The stepwise solid phase synthesis usually consists of repetitive cycles of α-amino group deprotection, washing, coupling (i.e., attachment of next amino acid residue to the growing peptide chain) and washing. After complete assembly of the peptide chain the terminal protecting group may be removed and, optionally, a labelling group may be coupled to the terminal amino group. The peptide may be cleaved from the resin support, side-chain protecting groups may be removed and the product may be purified following established methods of peptide chemistry.

In a further aspect the invention provides a method of screening for an inhibitor of ZAP-70 or Syk comprising

(a) incubating the ZAP-70 protein and/or the Syk protein with a compound of formula I, adenosine 5′-triphosphate (ATP), and a test compound; and

(b) assessing the ability of the test compound to moderate the interaction of the protein tyrosine kinases Syk and/or ZAP-70 with their substrate compared with the interaction resulting in the absence of the test compound.

The assessment step (b) is preferably performed by

(c) determining the amount of phosphorylated compound of formula I; and

(d) comparing the results with test compound to the results in the absence of a test compound (see examples C1-C4), a decrease in phosphorylation in the presence of a test compound indicating inhibition of ZAP-70 or Syk.

When ZAP-70 is used in step a), the method of the invention is useful to identify a ZAP-70 inhibitor, preferably an immunosuppressant, particularly an immunosuppressant specific to ZAP-70, more particularly an immunosuppressant which inhibits T-cell function, T-cell proliferation or natural killer cell function and natural killer cell proliferation.

When Syk is used in step a), the method of the invention is useful to identify a Syk inhibitor, preferably an immunosuppressant, particularly an immunosuppressant specific to Syk, more particularly an immunosuppressant which inhibits B-cell function and/or B-cell proliferation.

In accordance with the foregoing, the present invention further provides:

1. An assay to identify an inhibitor of ZAP-70 and/or Syk comprising

(a) incubating the ZAP-70 protein and/or the Syk protein with a compound of formula I, adenosine 5′-triphosphate (ATP), and a test compound; and

(b) assessing the ability of the test compound to moderate the interaction of the protein tyrosine kinases Syk and/or ZAP-70 with their substrate compared with the interaction resulting in the absence of the test compound.

2. A T cell inhibitor identified by the method or assay of the invention.

3. A NK cell inhibitor identified by the method or assay of the invention.

4. A B cell inhibitor identified by the method or assay of the invention.

5. An immunosuppressant identified by the method or assay of the invention.

6. A modulator of an inflammatory disease identified by the method or assay of the invention.

7. A method of inhibiting T-cell function and T-cell proliferation in a subject in need of such inhibition comprising administering to said subject an effective amount of an inhibitor to ZAP-70 and/or Syk identified by the method or assay of the invention.

8. A method of inhibiting NK-cell function and NK-cell proliferation in a subject in need of such inhibition comprising administering to said subject an effective amount of an inhibitor to ZAP-70 and/or Syk identified by the method or assay of the invention.

9. A method of inhibiting B-cell function and B-cell proliferation in a subject in need of such inhibition comprising administering to said subject an effective amount of an inhibitor to ZAP-70 and/or Syk identified by the method or assay of the invention.

10. A pharmaceutical composition comprising an inhibitor to ZAP-70 and/or Syk identified by the method or assay of the invention together with one or more pharmaceutically acceptable diluents or carriers therefore.

11. Use of a compound of formula I as a LAT substrate.

12. A compound of formula I which is phosphorylated, e.g. at least at one of the Tyr residues.

In the following examples all temperatures are in ° C. The following abbreviations are employed: DMF=dimethylformamide; Pmc=2,2,5,7,8-pentamethylchroman; tBu=tert.-butyl; DIPCDI=N,N′-diisopropylcarbodiimid; DIEA=N,N-diisopropyl-N-ethylamine; RT=room temperature; MS=molecular ion (e.g. M+H¹⁺) determined by electrospray mass spectroscopy; APC=allophycocyanine; ZAP-70=Zeta chain-associated protein of 70 kD; Syk=p72syk protein tyrosine kinase; IRK=insulin receptor kinase; SA=streptavidin; Lck=also called p56lck and has a relative molecular mass of 56,000 and belongs to the Src family of tyrosine kinases. It is expressed exclusively in lymphoid cells, predominantly in thymocytes and peripheral T cells.

### EXAMPLE A1

### Preparation of L(+)-biotinyl-aminohexanoyl-Glu-Glu-Gly-Ala-Pro-Asp-Tyr-Glu-Asn-Leu-Gln-Glu-Leu-Asn (SEQ ID NO:12)

The N-α Fmoc group of Fmoc-Asn(Trt)-oxymethyl-4-phenoxymethyl-co(polystyrene-1%-di-vinyl-benzene), content of Asn approx. 0.5 mmol/g, is cleaved using piperidine, 20% in DMF. Four equivalents per amino-group of Fmoc-amino acid protected in their side chains [Asp(OtBu), Glu(OtBu), Asn(Trt), Gln(Trt) and Tyr(tBu)] are coupled using DIPCDI and HOBt in DMF. After complete assembly of the peptide chain the terminal Fmoc-protecting group is removed with piperidine in DMF as before. L(+)-biotinyl-aminohexanoic acid is then coupled to the terminal amino group using DIPCDI and HOBt in DMF using four equivalents of the reagents for four days at RT. The peptide is cleaved from the resin support and all side-chain protecting groups are simultaneously removed by using a reagent consisting of 5% dodecylmethylsulfide and 5% water in TFA for two hours at RT. Resin particles are filtered off, washed with TFA and the product is precipitated from the combined filtrates by the addition of 10 to 20 volumes of diethyl ether, washed with ether and dried. The product is purified by chromatography on a C-18 wide-pore silica column using a gradient of acetonitrile in 2% aqueous phosphoric acid. Fractions containing the pure compound are collected, filtered through an anion-exchange resin (Biorad, AG4-X4 acetate form) and lyophilized to give the title compound. MS: 1958.0 (M−H)⁻¹

Using the procedure given for Example A1, but substituting the appropriate amino-acid derivatives and resin supports, compounds having the sequences of Table 1 are prepared:

TABLE 1

| A | | MS |
|---|---|---|
| 13 | R-Glu-Ser-Ile-Asp-Asp-Tyr- | 1673, 6 (M − H)¹⁻ |
| | Val-Asn-Val-Pro-Glu-Gly-OH | |
| 14 | R-Glu-Asp-Glu-Asp-Asp-Tyr- | 1586.9 (M + H)¹⁺ |
| | His-Asn-Pro-Gly-Gly-OH | |
| | Protected His (Trt) is used | |
| | in the assembly | |
| 15 | acetyl-Asp-Tyr-Glu-Asn-Leu- | 1177.9 (M − H)¹⁻ |
| | Gln-Glu-Leu-Asn-OH | |
| 16 | acetyl-Glu-Glu-Gly-Ala-Pro- | 1176 (M − H)¹⁻ |
| | Asp-Tyr-Glu-Asn-Leu-NH₂ | |
| | 4-(2′,4′-dimethoxyphenyl- | |
| | Fmoc-amino-methyl)-phenoxy- | |
| | co(polystyrene-1%-divinylben- | |
| | zene); acetylation after | |
| | cleavage of the terminal Fmoc | |
| | group. | |
| 17 | acetyl-Asp-Tyr-Glu-Asn-Leu- | 692 (M − H)¹⁻ |
| | NH₂ | |
| | 4-(2′,4′-dimethoxyphenyl- | |
| | Fmoc-amino-methyl)-phenoxy- | |
| | co(polystyrene-1%-divinylben- | |
| | zene); acetylation after | |
| | cleavage of the terminal Fmoc | |
| | group | |
| 18 | succinoyl-Glu-Glu-Gly-Ala- | 860 (M − 2H)²⁻ |
| | Pro-Asp-Tyr-Glu-Asn-Leu-Gln- | |
| | Glu-Leu-Asn-OH | |
| | succinoylation using succinic | |
| | anhydride and DIPEA in DMF | |
| | after removal of the terminal | |
| | Fmoc-group | |
| 19 | acetyl-Glu-Glu-Gly-Ala-Pro- | 847 (M − 2H)²⁻ |
| | Asp-Tyr-Glu-Phe-Leu-Gln-Glu- | |
| | Leu-Asn-OH | |
| 20 | R-Glu-Ser-Ile-Asp-Asp-Tyr- | 1736.4 (M − H)¹⁻ |
| | Glu-Asn-Met-Pro-Glu-Gly-OH | |
| 21 | acetyl-Glu-Glu-Gly-Ala-Pro- | 1661 (M − H)¹⁻ |
| | Asp-Tyr-Glu-Asn-Leu-Gln-Glu- | |
| | Leu-Asn-OH | |
| 22 | HOOC-CH₂-CH₂-CO-Asp-Tyr-Glu- | 750 (M − H)¹⁻ |
| | Asn-Leu-NH₂ | |
| | succinoylation as for SEQ ID | |
| | NO: 18 | |
| 23 | HOOC-CH₂-Asp-Tyr-Glu-Asn- | 708 (M − H)¹⁻ |
| | Leu-NH₂ | |
| | alkylation using bromoacetic | |
| | acid tert. butylate and DIPEA | |
| | in DMF after removal of the | |
| | terminal Fmoc-group | |
| 24 | HOOC-CH₂-CH₂-CO-Asp-Tyr-Glu- | 783 (M − H)¹⁻ |
| | Phe-Leu-NH₂ | |
| | succinoylation as for SEQ ID | |
| | NO: 18 | |
| 25 | R-Cys(H)-Asp-Asp-Tyr-Glu-Phe- | 1264.4 (M + Na)¹⁺ |
| | Leu-NH₂ | |

A: SEQ ID NO:
R: L(+)-biotinyl-aminohexanoyl

Compounds of formula I are useful as a component in a screening assay, in particular in a screening assay for identifying inhibitors of the protein tyrosine kinase ZAP-70 or of the protein tyrosine kinase Syk. These inhibitors which are capable of blocking protein tyrosine kinase ZAP-70 and/or protein tyrosine kinase Syk and therefore also blocking T cell activation and/or natural killer cell activation and/or B cell activation, find also use in treating disease associated with undesirable cell growth, differentiation, particularly immune cell differentiation, and hypersensitivity/allergy. In addition, they find use in the inhibition of autoreactive T cells, inhibition of production of deleterious T cell products (such as cytokines, lymphokines), T cell activities, Natural Killer cell activity and/or B-cell activity. These inhibitors are further useful as a modulator of an inflammatory disease. Thus, it could be relevant in the prevention and treatment of immunological disease, e.g. autoimmune disease, T-cell immunomodulation, and graft rejection, but also in cancer where cell signaling, in which ZAP-70 and Syk play a role, is disturbed.

For therapeutic uses, the inhibitory compositions of the invention can be administered as pharmaceutical compositions. Such pharmaceutical compositions for use in accordance with the present invention may be formulated in conventional manner using one or more physiologically acceptable carriers or excipients. Thus, the compounds and their physiologically acceptable salts and solvates may be formulated for administration by inhalation or insufflation (either through the mouth or the nose) or topical, oral, buccal, parenteral or rectal administration.

ZAP-70 protein or Syk protein may be prepared from a natural source or by applying genetic engineering techniques involving e.g. preparing suitable primers and screen for nucleic acid comprising the nucleotide sequence encoding ZAP-70 or Syk in a library made up of genomic DNA or cDNA, prepare multiple copies of said nucleic acid and insert said nucleic acid into suitable vectors for introduction into suitable host cells, e.g. baculovirus, or directly introduce the nucleic acid into host cells, cultivate the resulting host cells and purify the desired enzyme.

The following examples further illustrate the present invention, and the examples are provided for illustration purposes and are not intended to be limiting the invention.

### EXAMPLE B1

### Cloning, Expression and Purification of Recombinant ZAP-70 Kinase

A nucleic acid encoding full-length human ZAP-70 (GenBank #L05148) is amplified from a Jurkat cDNA library by RT-PCR and cloned into the pBluescript KS vector (Stratagene, Ca.). The authenticity of the ZAP-70 cDNA insert is validated by complete sequence analysis. This donor plasmid is then used to construct a recombinant baculovirus transfer vector based on the plasmid pVL1392 (Pharmingen, Ca.) featuring in addition an N-terminal hexa-histidine tag. Following co-transfection with AcNPV viral DNA, ten independent viral isolates are derived via plaque-purification, amplified on small scale and subsequently analysed for recombinant ZAP-70 expression by Western Blot using a commercially available anti-ZAP-70 antibody (Clone 2F3.1, Upstate Biotechnology). Upon further amplification of one positive recombinant plaque, titrated virus stocks are prepared and used for infection of Sf9 cells grown in serum-free SF900 II medium (Life Technologies) under defined, optimised conditions.

ZAP-70 protein is isolated from the lysate of infected Sf9 cells by affinity chromatography on a Ni-NTA column.

Recombinant full-length human Syk (GenBank #Z29630), full-length human Bruton's tyrosine kinase (Btk) (EMBL #X58957), full-length human Lck (GenBank #X06369), and the cytoplasmic domain of human IRK (GenBank #M10051) are produced by analogous methods.

Assays to determine the amount of phosphorylation of suitable substrates by ZAP-70 or Syk may involve the following techniques: Utilizing radiolabelled ATP, e.g. containing ³³P, radioactivity incorporated into compounds of formula I may be determined by scintillation counting. Methods for separation of the phosphorylated products from the remaining ³³P ATP include capture on phosphocellulose or membrane-linked streptavidin, or polyacrylamide gel electrophoresis. Homogenous assays, not requiring a separation step, like scintillation proximity methods use scintillants incorporated into a solid phase for detection.

Non-radioactive techniques may be based on enzyme immunoassays where anti-phosphotyrosine antibodies are used to quantify phosphorylation levels of immobilized compounds of formula I. Furthermore, fluorescence detection methods may be used, e.g. fluorescence polarisation, fluorescence resonance energy transfer, time resolved fluorescence, time resolved fluorescence resonance energy transfer, fluorescence correlation spectroscopy. Tyrosine kinase activity may be assayed using fluorescein-conjugated anti-phosphotyrosine antibodies.

### EXAMPLE C1

### Phosphorylation of Compounds of Formula I by ZAP-70 Measured by Time Resolved Fluorescence Resonance Energy Transfer

ZAP-70 kinase assay: 10 nM ZAP-70 alone or 10 nM ZAP-70 plus 42 nM Lck, 250 nM biotinylated compound of formula I and 1 μM ATP are incubated in 40 μl ZAP-70 kinase buffer (20 mM Tris, pH 7.5, 10 μM Na₃VO₄, 1 mM DTT, 1 mM MnCl₂, 0.01% bovine serum albumin, 0.05% Tween 20) in microtiter plates for 5 hours at RT. The kinase reaction is terminated with 10 μl of a 10 mM EDTA solution in detection buffer (20 mM Tris, pH 7.5, 0.01% bovine serum albumin, 0.05% Tween 20).

The detection phase is performed by addition of 50 μl europium-labelled anti-phosphotyrosine antibody (e.g. EU-PT66; final concentration 0.125 nM; Advant/Wallac) and 50 μl streptavidin-allophycocyanine (SA-APC; final concentration 40 nM) in detection buffer. After 1 hour incubation at RT fluorescence is measured, e.g., on the Victor2 Multilabel Counter (Wallac) at 665 nm.

In this assay compounds of formula I, in particular compounds represented by SEQ ID NO:12, SEQ ID NO:13, SEQ ID NO:14 or SEQ ID NO:25 are phosphorylated by ZAP-70 and by ZAP-70 activated by Lck resulting in 20,000 to 46,000 fluorescence units.

### EXAMPLE C2

### Phosphorylation of Compounds of Formula I by ZAP-70 and ³³P-ATP

ZAP-70 (20 nM) is activated in vitro with Lck (100 nM) and ATP (10 μM) in 15 μl ZAP-70 kinase buffer. The reaction is performed in siliconized microtiter plates for one hour at RT. After addition of 5 μl ZAP-70 kinase buffer containing 5 μM of the selective Lck inhibitor PP2 (4-amino-5-(4-chloro-phenyl)-7-(t-butyl)pyrazolo[3,4-d]pyrimidine; Alexis Biochemicals; final concentration 1 μM) and 30 nM ³³P-ATP (110 Tbq/mmol) the mixture is incubated for further 10 min at RT.

Then, 0.4-100 μM of a compound of formula I (final concentration 0.08-20 μM) in 5 μl ZAP-70 kinase buffer are added and incubated at RT for 2 hours. The kinase reaction is terminated by addition of 12.5 μl 7.5 M guanidine hydrochloride.

Thirty μl are transferred to an individual SAM²™ biotin capture plate well (Promega) which had been incubated with 100 μl per well phosphate-buffered saline. After an incubation of 1 min washing is performed in a vacuum manifold system as follows: 5 times with 200 μl 2 M NaCl per well, 6 times with 200 μl 2 M NaCl/1% H₃PO₄ per well, 2 times with 200 μl deionized water per well, 2 times with 200 μl 95% ethanol per well. The plates are air dried and an opaque seal is applied to the bottom of the plate, 15 μl per well scintillation fluid (e.g. Microscint, Packard) is added and the plate is covered with a transparent top seal. Radioactivity is determined with a liquid scintillation spectrometer (e.g. TopCount, Packard). E.g. compounds of SEQ ID NO:12; SEQ ID NO: 13; or SEQ ID NO: 14 are phosphorylated by ZAP-70. Maximal cpm range from 12,000 to 16,000. Kₘ values are in the range of from 0.4 to 2.0 μM.

### EXAMPLE C3

### Phosphorylation of Compounds of Formula I by Syk Measured by Time Resolved Fluorescence Resonance Energy Transfer

Syk kinase assay: 10 nM Syk, 370 nM compounds of formula I and 20 μM ATP are mixed in 40 μl Syk Kinase buffer (20 mM Tris, pH 7.5, 10 μM Na₃VO₄, 1 mM DTT, 10 mM MgCl₂, 0.01% bovine serum albumin, 0.05% Tween 20) and incubated for 4 hours at RT. Termination of the kinase reaction, detection phase and determination of fluorescence is performed as described in Example C1.

In this assay compounds of formula I, in particular compounds represented by SEQ ID NO:12, SEQ ID NO:13 or SEQ ID NO:14 are phosphorylated by Syk resulting in 20,000 to 30,000 fluorescence units.

### EXAMPLE C4

### Lack of Phosphorylation of Compounds of Formula I by Lck, Btk or IRK Measured by Time Resolved Fluorescence Resonance Energy Transfer

Lck assay: 42 nM Lck, 250 nM compounds of formula I and 1 μM ATP are incubated in 40 μl ZAP-70 kinase buffer (see Example C1). Termination of the kinase reaction, detection phase and determination of fluorescence is performed as described in Example C1.

Btk assay: 50 nM Btk, 370 nM compound of formula I and 20 μM ATP are mixed in 40 μl Btk buffer (20 mM Tris, pH 7.5, 10 μM Na₃VO₄, 1 mM DTT, 10 mM MnCl₂, 2 mM MgCl₂, 0.01% bovine serum albumin, 0.05% Tween 20) and incubated for 1 hour at RT. Eu-PT66 (2 nM) and SA-APC (80 nM) in 100 μl detection buffer added and the fluorescence is determined after one hour as described in Example C1.

IRK assay: 0.3 nM IRK, 120 nM compound of formula I and 20 μM ATP are incubated in 40 μl IRK buffer (20 mM Tris, pH 7.5, 10 μM Na₃VO₄, 1 mM DTT, 2 mM MnCl₂, 20 mM MgCl₂, 0.01% bovine serum albumin, 0.05% Tween 20) for 5 hours at RT.

Termination of the kinase reaction, detection phase and determination of fluorescence is performed as described in Example C1.

In this assay compounds of formula I, e.g. compounds with SEQ ID NO:12; SEQ ID NO: 13; and SEQ ID NO:14, are hardly phosphorylated by Lck, Btk or IRK resulting in 1,300 to 5,600 fluorescence units (vs. 20,000 to 46,000 fluorescence units in Example C1).

### EXAMPLE C5

### Phosphorylation of Compounds of Formula I by ZAP-70 Measured in a Competitive Assay

80 nM ZAP-70 are incubated with 80 nM Lck and 4 μM ATP in ZAP-70 kinase buffer for 1 hour at RT in a siliconized polypropylene tube. Then, the selective Lck inhibitor PP2 (see Example C2) is added (final concentration 1.2 μM) and incubated for further 10 min.

Ten μl of this solution is mixed with 10 μl biotinylated compound of e.g. SEQ ID NO:12 (1 μM) and 20 μl of the non-biotinylated peptides of compounds of formula I, e.g. by SEQ ID NO:21, SEQ ID NO:15, SEQ ID NO:16 or SEQ ID NO:17 (serial dilution 0.28 to 200 μM) and incubated for 4 hours at RT. Termination of the kinase reaction, detection phase and determination of fluorescence is performed as described in Example C1.

In this assay compounds of formula I, e.g. compounds represented by SEQ ID NO:21, SEQ ID NO:15, SEQ ID NO:16, SEQ ID NO:17, SEQ ID NO:22, SEQ ID NO:23 or SEQ ID NO:24 inhibit the phosphorylation of the compound represented by SEQ ID NO:12 by ZAP-70. The IC₅₀ values are in the range of from 0.6 to 21 μM.

1. A compound of formula I
R¹-Tyr-R² (I)
wherein
R¹ is HOOC(CH₂)₂CO— or R³-Asp- wherein R³ is hydrogen, an amino acyl residue or an oligopeptidyl residue; and
R² is an amino acid or an oligopeptide;
with the proviso that compounds represented by SEQ ID NO:1, SEQ ID NO:2, SEQ ID NO:3, and SEQ ID NO:4 are excluded.

2. A compound of formula IIa or IIb
R⁴—Y¹—(X⁶)ₐ(X⁵)_{b}(X₄)_{c}(X³)_{d}(X²)ₑAspTyrX^{1′}(X^{2′})ᵣ(X^{3′)}_{q}(X^{4′})ₚ(X^{5′})ₒ(X^{6′})ₙ—NHCH(R⁶C(O)—R⁵ (IIa)
HOOC(CH₂)₂CO-Tyr-X^{1′}—(X^{2′})ᵣ—(X^{3′})_{q}—(X⁴)ₚ—(X^{5′})ₒ—(X^{6′)}ₙ—NHCH(R⁶)C(O)—R⁵ (IIb)
wherein
each of X⁶, X⁵, X⁴, X³, X², X^{1′}, X^{2′}, X^{3′}, X^{4′}, X^{5′}and X^{6′} is an amino acid;
Y¹ is a linking group or single bond;
R⁴ is hydrogen, a labelling group, C₁-C₈alkoxycarbonyl, R^{a}—C(O)—, R^{a}—OC(O)— or R^{a}—NHC(O)— wherein R^{a} is linear or branched C₁-C₈alkyl, carboxy-C₁-C₈alkyl, aryl or aralkyl wherein the aryl group is unsubstituted or substituted by halogen, C₁-C₄alkyl or C₁-C₈alkoxy;
R⁵ is NR⁷R⁸ or OR⁹ wherein R⁷ and R⁸ independently are hydrogen, C₁-C₈alkyl, or R⁷ and R⁸ together with the nitrogen atom to which they are attached form a heterocyclic residue, and R⁹ is hydrogen or C₁-C₈alkyl;
R⁶ is an amino acid side chain; and
(a) a, b, c, d and e are 1;
(b) a is 0 and b, c, d and e are 1;
(c) a and b are 0 and c, d and e are 1;
(d) a, b and c are 0 and d and e are 1;
(e) a, b, c and d are 0 and e is 1; or
(f) a, b, c, d and e are 0; and
(a′) n, o, p, q and r are 1;
(b′) n is 0 and o, p, q and r are 1;
(c′) n and o are 0 and p, q and r are 1;
(d′) n, o and p are 0 and q and r are 1;
(e′) n, o, p and q are 0 and r is 1; or
(f′) n, o, p, q and r are 0.

3. A compound represented by SEQ ID NO:21, wherein Xaa is H₃CC(O)—, and SEQ ID NO:12, wherein Xaa is L(+)-biotinyl-aminohexanoyl and SEQ ID NO: 25 wherein Xaa is L(+)-biotinyl-aminohexanoyl.

4. A method of screening for an inhibitor of ZAP-70 or Syk comprising
(a) incubating the ZAP-70 protein and/or the Syk protein with a compound of formula I, adenosine 5′-triphosphate (ATP), and a test compound; and
(b) assessing the ability of the test compound to moderate the interaction of the protein tyrosine kinases Syk and/or ZAP-70 with their substrate compared with the interaction resulting in the absence of the test compound.

5. An assay to identify an inhibitor to ZAP-70 and/or Syk comprising
(a) incubating the ZAP-70 protein and/or the Syk protein with a compound of formula I, adenosine 5′-triphosphate (ATP), and a test compound; and
(b) assessing the ability of the test compound to moderate the interaction of the protein tyrosine kinases Syk and/or ZAP-70 with their substrate compared with the interaction resulting in the absence of the test compound.

6. An immunosuppressant specific for ZAP-70 and/or Syk identified by a method according to claim 4 or an assay according to claim 5.

7. An immunosuppressant specific for T cells or specific for T cells and NK cells identified by a method according to claim 4 or an assay according to claim 5.

8. A T cell inhibitor and/or a B cell inhibitor and/or NK cell identified by a method according to claim 4 or an assay according to claim 5.

9. A modulator of an inflammatory disease identified by a method according to claim 4 or an assay according to claim 5.

10. A method of inhibiting T-cell function and T-cell proliferation in a subject in need of such inhibition comprising administering to said subject an effective amount of an inhibitor to ZAP-70 and/or Syk identified by a method according to claim 4 or an assay according to claim 5.

11. A method of inhibiting NK-cell function and NK-cell proliferation in a subject in need of such inhibition comprising administering to said subject an effective amount of an inhibitor to ZAP-70 and/or Syk identified according to claim 4 or an assay according to claim 5.


SEQUENCE LISTING (25 sequences)

SEQ ID NO: 1 - protein, 14 aa, Artificial
  PEPTIDE (1)..(14): Peptide derived from human source
SFEEDDYESPNDDQ

SEQ ID NO: 2 - protein, 13 aa, Artificial
  PEPTIDE (1)..(13): Peptide derived from human source
EELQDDYEDMMEE

SEQ ID NO: 3 - protein, 15 aa, Artificial
  PEPTIDE (1)..(15): Peptide derived from human source
GEDDGDDYESPNEEE

SEQ ID NO: 4 - protein, 12 aa, Artificial
  PEPTIDE (1)..(12): Peptide derived from human source
ALEKDDYEEVGV

SEQ ID NO: 5 - protein, 13 aa, Artificial
  PEPTIDE (1)..(13): Peptide derived from human source Glu at N-terminal has R4-Y1 attached Leu at C-terminal has -NHCH(R6)C(O)-R5 attached
EEGAPDYENLQEL

SEQ ID NO: 6 - protein, 11 aa, Artificial
  PEPTIDE (1)..(11): Peptide derived from human source Glu at N-terminal has R4-Y1 attached Glu at C-terminal has -NHCH(R6)C(O)-R5 attached
ESIDDYVNVPE

SEQ ID NO: 7 - protein, 10 aa, Artificial
  PEPTIDE (1)..(10): Peptide derived from human source Glu at N-terminal has R4-Y1 attached Gly at C-terminal has -NHCH(R6)C(O)-R5 attached
EDEDDYHNPG

SEQ ID NO: 8 - protein, 8 aa, Artificial
  PEPTIDE (1)..(8): Peptide derived from human source Asp at N-terminal has R4-Y1 attached Leu at C-terminal has -NHCH(R6)C(O)-R5 attached
DYENLQEL

SEQ ID NO: 9 - protein, 9 aa, Artificial
  PEPTIDE (1)..(9): Peptide derived from human source Glu at N-terminal has R4-Y1 attached Asn at C-terminal has -NHCH(R6)C(O)-R5 attached
EEGAPDYEN

SEQ ID NO: 10 - protein, 4 aa, Artificial
  PEPTIDE (1)..(4): Peptide derived from human source Asp at N-terminal has R4-Y1 attached Asn at C-terminal has -NHCH(R6)C(O)-R5 attached
DYEN

SEQ ID NO: 11 - protein, 14 aa, Artificial
  PEPTIDE (1)..(14): Peptide derived from human source Glu at N-terminal has R4-Y1 attached
EEGAPDYENLQELN

SEQ ID NO: 12 - protein, 14 aa, Artificial
  PEPTIDE (1)..(14): Peptide derived from human source Glu at N-terminal has H3CC(O)- attached
EEGAPDYENLQELN

SEQ ID NO: 13 - protein, 12 aa, Artificial
  PEPTIDE (1)..(12): Peptide derived from human source Glu at N-terminal has L(+)-biotinyl-aminohexanoyl attached
ESIDDYVNVPEG

SEQ ID NO: 14 - protein, 11 aa, Artificial
  PEPTIDE (1)..(11): Peptide derived from human source Glu at N-terminal has L(+)-biotinyl-aminohexanoyl attached
EDEDDYHNPGG

SEQ ID NO: 15 - protein, 9 aa, Artificial
  PEPTIDE (1)..(9): Peptide derived from human source Asp at N-terminal has L(+)-biotinyl-aminohexanoyl attached
DYENLQELN

SEQ ID NO: 16 - protein, 10 aa, Artificial
  PEPTIDE (1)..(10): Peptide derived from human source Glu at N-terminal has acetyl attached
EEGAPDYENL

SEQ ID NO: 17 - protein, 5 aa, Artificial
  PEPTIDE (1)..(5): Peptide derived from human source Asp at N-terminal has acetyl attached
DYENL

SEQ ID NO: 18 - protein, 14 aa, Artificial
  PEPTIDE (1)..(14): Peptide derived from human source Glu at N-terminal has acetyl attached
EEGAPDYENLQELN

SEQ ID NO: 19 - protein, 14 aa, Artificial
  PEPTIDE (1)..(14): Peptide derived from human source Glu at N-terminal has succinoyl attached
EEGAPDYEFLQELN

SEQ ID NO: 20 - protein, 12 aa, Artificial
  PEPTIDE (1)..(12): Peptide derived from human source Glu at N-terminal has acetyl attached
ESIDDYENMPEG

SEQ ID NO: 21 - protein, 14 aa, Artificial
  PEPTIDE (1)..(14): Peptide derived from human source Glu at N-terminal has L(+)-biotinyl-aminohexanoyl attached
EEGAPDYENLQELN

SEQ ID NO: 22 - protein, 5 aa, Artificial
  PEPTIDE (1)..(5): Peptide derived from human source Asp at N-terminal has acetyl attached
DYENL

SEQ ID NO: 23 - protein, 5 aa, Artificial
  PEPTIDE (1)..(5): Peptide derived from human source Asp at N-terminal has HOOC-CH2-CH2-CO- attached
DYENL

SEQ ID NO: 24 - protein, 5 aa, Artificial
  PEPTIDE (1)..(5): Peptide derived from human source Asp at N-terminal has HOOC-CH2- attached
DYEFL

SEQ ID NO: 25 - protein, 7 aa, Artificial
  PEPTIDE (1)..(7): Peptide derived from human source Cys at N-terminal has HOOC-CH2-CH2-CO- attached
CDDYEFL